(19) United States
(12) Patent Application Publication
(10) Pub. No.: US 2002/0087036 A1
(43) Pub. Date: Jul. 4, 2002

(54) Process for the catalytic hydrogenation of organic compounds and supported catalyst therefor

(76) Inventors: Thomas Haas, Frankfurt (DE); Bernd Jaeger, Darmstadt (DE); Jorg Sauer, Mobile, AL (US); Rudolf Vanheertum, Brasschaat (BE)

Correspondence Address: SMITH, GAMBRELL & RUSSELL, LLP, ATTORNEYS AT LAW, SUITE 800, 1850 M STREET, N.W., WASHINGTON, DC, 20036, US

(21) Appl. No.: 09/985,179

(22) Filed: Nov. 1, 2001

(30) Foreign Application Priority Data
Nov. 2, 2000 (DE) 100 54 347.2

(51) Int. Cl. C07C 27/04; C 07C 2 9/30

(52) U.S. Cl. 568/885; 564/423

(57) ABSTRACT

A process for the catalytic hydrogenation of an organic compound, in particular a labile organic compound, in the presence of a support catalyst with a coating containing ruthenium as active metal and a total of 1.01 to 30 wt. % of active metals. Higher stereoselectivity and a greater catalyst shelf life may be obtained by using a support catalyst of which the oxide, carbide, nitride or siliceous support material has a BET (N₂) surface area smaller than 10 m²/g, particularly preferably 0.1 to 5 m²/g, prior to loading with at least one active metal diatomaceous earth with a BET (N₂) surface area greater than 2 m²/g is excluded and the ruthenium content thereof makes up at least 50 wt. %, preferably at least 99 wt. % of the active metals. The process and the catalysts are particularly suitable for the hydrogenation of polyfunctional compounds such as hydroxycarbonyl compounds and aromatic amines.

### INTRODUCTION AND BACKGROUND

The present invention relates to a process for the catalytic hydrogenation of organic compounds, in particular labile organic compounds, in the presence of a supported catalyst with a coating containing ruthenium as active metal. The term “labile compounds” as used herein denotes compounds containing more than one hydrogenatable grouping, or at least one further functional group in addition to a hydrogenatable grouping. In a further aspect, the invention also relates to supported catalysts containing ruthenium as active metal, which are suitable for carrying out the process and with which high selectivity and stereoselectivity can be achieved.

It is known to hydrogenate organic compounds with one or more unsaturated compounds and optionally additional functional groups using a noble metal-containing supported catalyst. The result of hydrogenation, in particular the selectivity and stereoselectivity, depend on the structure of the support material in addition to the catalytically active noble metal.

According to U.S. Pat. No. 4,343,955, alkyl phenols may be hydrogenated in the presence of a supported catalyst based on ruthenium on an aluminum oxide support to form alkyl cyclohexanols with a high cis content. The aluminum oxide used to produce the supported catalyst must have a sufficiently great specific surface area, in particular 100 to 300 m²/g. The corresponding alkyl benzene is also formed to a certain extent by dehydrogenation as a by-product. However, greater stereoselectivity than in this document is desired in many cases simultaneously with a long shelf life of the catalyst.

EP 0 814 098 teaches a process for reacting various organic compounds, including aromatic compounds, in which at least one hydroxyl group or one amino group is bound to the aromatic nucleus, also carbonyl compounds, nitrites and multiple unsaturated polymers in the presence of a support-type ruthenium catalyst. High conversion rates and yields as well as high catalyst loading and long shelf lives are achieved if 10 to 50% of the pore volume of the support is formed by macropores with a pore diameter of 50 nm to 10,000 nm and 50 to 90% of the pore volume of the support is formed by mesopores with a pore diameter in the range of 2 to 50 nm. The support, which is activated carbon or oxide or carbide materials, preferably has a BET surface area of 50 to 500m²/g. Although catalysts of this type have high hydrogenation activity, the stereoselectivity of a process carried out with them is slight, as shown by example 3 of this document—trans-4-tert.-butylcyclohexanol and its cis isomers are formed in a ratio of 2 to 1 during the hydrogenation of p-tert.-butylphenol.

As shown by WO 98/57913, for the hydrogenation of labile educts such as, for example, 3-hydroxypropanal to 1,3-propane diol, a Ru-activated carbon support catalyst is unsuitable for an industrial process despite a high mesopore content and a surface area of about/over 200 m²/g, because these catalysts deactivate very rapidly. Higher selectivity and shelf lives of the catalysts are achieved by using oxide supports. However, a low hydrogenation temperature and therefore also low catalyst activity have to be accommodated.

According to U.S. Pat. 5,110,779, a supported catalyst based on a macroporous support material such as diatomaceous earth, aluminum oxide or activated carbon with a coating of a metal from group VIII, such as palladium, platinum and ruthenium, is suitable for the hydrogenation of unsaturated polymers. The pore distribution is an important characteristic of the supported catalyst and essential for good olefin hydrogenation, 90% of the pore volume consisting of pores having a diameter of >1,000 Angstrom and the ratio of the metal surface to the support surface lying in the range of 0.07 to 0.75:1. The support materials used in the examples had the following BET surface area: diatomaceous earth 2.5 to 3.5 m²/g; Al₂O₃ 10 to 15 m²/g; activated carbon 6 to 10 m²/g. In comparison, aluminum oxide and silica with a BET surface area in the range of 30 to about 300 m²/g were found to have minimal catalytic activity during the hydrogenation of olefinic polymers.

Ruthenium on aluminum oxide or titanium dioxide is used as catalyst in the process described in DE patent application 19,942,813 for the production of 4,4′-diaminodicyclohexylmethane by hydrogenation of methylenedianiline as catalyst. The BET surface area of the TiO₂ is given as 40 to 50 m²/g and that of the Al₂O₃ is about 230 m²/g. A drawback of this process is the requirement of having to use a supported catalyst with a very high ruthenium content.

It is accordingly an object of the invention to provide a process for the hydrogenation of organic compounds, in particular labile organic compounds, in the presence of a ruthenium supported catalyst, which is better than the previously known processes.

According to a further object of the invention, the hydrogenation of compounds which may lead to stereoisomers should be adapted to be carried out with higher stereoselectivity.

According to a still further object, the catalyst activity during the hydrogenation of labile compounds such as hydroxyalkanals and aliphatic dinitriles should be higher than in previously known processes, and, in addition, the content of by-products should not increase with the shelf life of the catalyst.

### SUMMARY OF THE INVENTION

The aforementioned objects and further objects, which will emerge from the flowing description, can be achieved by the process according to the invention and by the catalyst used for carrying out the process.

A process has been found for the catalytic hydrogenation of an organic compound, in particular a labile organic compound, in the presence of a supported catalyst with a coating containing ruthenium as the support-bond active metal and a total of 1.01 to 30 wt. % of active metals, which is characterized in that a supported catalyst is used, of which the oxide, carbide, nitride or siliceous support material has a BET (N₂) surface area smaller than 10 m²/g prior to loading with at least one active metal and of which the ruthenium content makes up at least 50 wt. % of the active metals. In the event that diatoaceous earth is used as the support, the BET (N₂) surface area cannot be greater than 2m²/g.

In a preferred practical example, the catalyst contains at least 90 wt. %, preferably at least 99 wt. % of ruthenium as the active metal. The supported catalyst can contain one or more other catalytically active metals, in particular metals from the 1st, 7th and 8th subsidiary group of the Periodic Table of Element as active metals, in addition to at least 50 wt. % of ruthenium. These other metals are, in particular, palladium, platinum, copper, cobalt and nickel.

The support materials to be used for producing the supported catalysts to be used according to the invention are an oxide, nitride, carbide or siliceous material; however, diatomaceous earths with a BET (N₂) specific surface area of >2 m²/g are excluded. Suitable oxide materials include, in particular, naturally occurring and synthetically produced oxides of aluminum, silicon, titanium, zirconium, magnesium, zinc and mixtures thereof or mixed crystals such as perovskites, for example MgAl₂O₄. Aluminum oxide, titanium dioxide, silicon dioxide and zirconium dioxide are particularly suitable oxide materials. The siliceous support materials include, in particular, synthetic aluminum silicates as well as zirconium silicates. The nitride support materials include, in particular, nitrides of aluminum, silicon, titanium, zirconium, niobium and tantalum, as well as nitrides which contain at least one of these metals and additionally a further metal. Carbides such as silicon carbide may also be used. Glass frits having various compositions are also to be included as oxide or siliceous support materials.

### DETAILED DESCRIPTION OF INVENTION

According to the invention, the support materials of the supported catalysts to be used according to the invention have a specific surface area, measured by the BET method by N₂ adsorption, for example to DIN 66131, of <10 m²/g, in particular equal to or smaller than 5 m²/g and particularly preferably 0.1 to 2 m²/g. Thus, the broad range is 0.1 to <10 m²/g. Supported catalysts, conventional in the state of the art and of which the support material has a specific surface area in the range of 50 to 500 m²/g—see for example EP 0 814 098—were used for hydrogenation, dehydrogenation, hydrogenolysis and aminating hydrogenation. There are only a few documents relating to hydrogenation using ruthenium-containing oxide supported catalysts of which the support material contains 10 or 20 m²/g in the state of the art—see U.S. Pat. No. 5,110,779 and DE 199 42 813. These documents relate to the hydrogenation of quite specific compounds but, with the exception of certain diatomaceous earths, do not show a support material having a BET surface area of <10 m²/g, in particular <5 m²/g and particularly preferably 0.1 to 2 m²/g for ruthenium-containing support catalysts. Support materials with the BET surface area according to the invention may be obtained, for example, by known precipitation processes or flame-pyrolitic processes, production being followed by a calcination stage; the desired BET surface areas may be adjusted as a function of the calcination temperature and calcination period. Some of the support materials to be used according to the invention are naturally occurring products such as, for example, quartz, rutile and zirconium. The aforementioned support materials are coated with ruthenium and optionally additionally further metals in a manner known per se. The so-called “incipient wetness method”(published in Preparation of Catalysts, Delmond, B., Jakobs, P. A., Poncalt, G., Amsterdam Elsevier, 1976, page 13) is particularly suitable. With this method, the water adsorption capacity of the support material is initially determined. Depending on this adsorption capacity, an aqueous ruthenium chloride solution or a solution also containing compounds of other metals having hydrogenation action apart from ruthenium chloride is produced in the concentration required for the coating. The support is then treated with this solution, the entirety of the solution being adsorbed. The loaded support is dried under normal pressure or reduced pressure in an inert gas atmosphere preferably at 20 to 100° C. The impregnated support is finally hydrogenated to form the metals having hydrogenation action, preferably using hydrogen at a temperature of 100 to 500° C. for a period of 20 minutes to 24 hours. If desired, the hydrogenated supported catalyst is washed out. The aforementioned method of preparation leads to a fine distribution of the ruthenium on the catalyst support, the crystallite size generally being in the range of 1 to 5 nm. The loading of the support material with ruthenium or ruthenium with other metals having hydrogenation action usually lies in the range of 0.1 to 20 wt. %, in particular 0.1 to 10 wt. %. According to a preferred practical example, the supported catalyst contains 0.1 to 5 wt. %, in particular 0.5 to 3 wt. % of ruthenium or ruthenium with other active metals, ruthenium preferably making up more than 90%, in particular more than 99% of the active metals.

Unsaturated organic compounds such as olefins, aromatic compounds, aldehydes, ketones, esters, carboxylic acid amides, imines and nitrites may be hydrogenated using the ruthenium-containing support catalysts according to the invention. The special structure of the ruthenium-containing support catalyst to be used makes it particularly suitable for the hydrogenation of labile compounds. The term labile compounds here includes those which contain one or more functional groups in addition to the unsaturated grouping to be hydrogenated and which can therefore reduce the selectivity of hydrogenation by subsequent or secondary reactions, so one or more other products are also formed in addition to the hydrogenated target product. The organic compounds to be hydrogenated are labile, in particular, if the functional group is in the α-, β- or γ-position to the unsaturated grouping. The functional group may be a hydrogenatable functional group such as a carbonyl or nitrile group.

Examples of organic compounds which respond to the hydrogenation according to the invention include: aromatic and heteroaromatic amines such as 4,4′-, 2,2′- and 2,4′-diaminodiphenylmethane and isomeric mixtures thereof; aromatic and aliphatic nitrites such as substituted benzonitriles and nicotinic acid nitrile; alkylated or otherwise substituted phenols such as tertiary butylated phenols, bisphenols and alcoxylated phenols, the term “phenols” also covering polynuclear aromatic systems with one or more hydroxyl groups on the aromatic substance; aromatic dicarboxylic acid esters such as dimethylterephthalate.

Examples from the series of hydrogenatable organic compounds, in particular hydrogenatable labile organic compounds, include: hydroxycarbonyl compounds in which the hydroxyl group is in the α-, β- or γ-position such as 3-hydroxypropanal and carbohydrates such as glucose; aliphatic or cycloaliphatic dinitriles such as ethylene dinitrile; aliphatic and cycloaliphatic aldehydes and ketones which additionally contain a nitrile group such as isophorone nitrile or isophorone imine in the β- or γ-position.

Hydrogenation is carried out in a manner known per se in that the organic compound to be hydrogenated is reacted in the presence or absence of a solvent at ambient temperature or elevated temperature under corresponding hydrogen pressure in the presence of the supported catalyst according to the invention. The catalyst may be used here in the form of a suspension catalyst or a fixed-bed catalyst.

Fixed-bed hydrogenation may be carried out by the so-called bubble mode of operation (flooded fixed-bed) or by the trickle bed mode of operation. The trickle bed mode of operation, in which a liquid medium containing the organic compound trickles over the catalyst bed, and hydrogen flows in a co-current or counter-current thereto, is preferred in particular during the hydrogenation of labile organic compounds.

It has been found that the support catalysts to be used according to the invention surprisingly lead to higher stereoselectively of the reaction than ruthenium support catalysts based on a support material with a BET surface area of >10 m²/g. At the same time, it has been found that the catalyst activity of supported catalysts to be used according to the invention remains constant even after a prolonged period of operation, so the product composition of the hydrogenated product also remains substantially constant. When using previously known Ru-support catalysts, the proportion of by-products increases and the catalyst shelf life decreases as the operating period increases. It is assumed that the aforementioned advantages are due to the fact that, owing to the catalyst structure, the organic compound to be hydrogenated is only temporarily in contact with the catalytically active surface as it is not held in mesopores.

The Advantages of the Invention are

the new Ru support catalysts are particularly suitable for the hydrogenation of labile compounds;

the catalysts are distinguished by a long shelf life;

the composition of the hydrogenated product remains substantially constant even after a prolonged operating period;

polyfunctional organic compounds of higher stereoselectivity may be obtained in the presence of the Ru support catalyst according to the invention.

The following examples and comparison examples illustrate the process according to the invention and the advantages achievable thereby.

Production of the Catalyst

The water uptake of the support was determined in g of H₂O per 100 g of support.

RuCl₃ was dissolved in distilled water in order to load 250 ml of support. 250 ml were placed in a coating pan and were moistened with the RuCl₃ solution while the pan rotated.

The coated support was dried for 16 hours in air, then heated to 200° C. in a tubular furnace. The coated support was then reduced for 8 hours at 200° C. using hydrogen. The reduced Ru supported catalyst was washed free of chloride three times with 40 ml of distilled water in each case. The following table shows important features of two Ru supported catalysts according to the invention (examples 1 and 2) as well as the features of two Ru supported catalysts not in accordance with the invention (comparison examples 1 and 2).

| | | BET (N₂) | Support | Ru-loading |
|---|---|---|---|---|
| | Support | g/m² support | d (mm) | (wt. %) |
| B1 | α Al₂O₃ | approx. 0.2 | 1.5 | 2.0 |
| B2 | rutile | 1 | 1.5 | 1.6 |
| VB1 | γ Al₂O₃ | 230 | 1.2 | 5.0 |
| VB1 | rutile/anatase | 52 | 1 | 1.2 |

B = example; VB = comparison example
d = diameter

### EXAMPLE 3

4,4′-methylenedianiline (=4,4′-HMDA) was hydrogenated to 4,4′-diamino-dicyclohexylmethane (4,4′-HMDA) with a low trans-trans-isomer content.

Hydrogenation was carried out continuously in a trickle bed apparatus with a reactor volume of 45 ml. The apparatus consisted of a liquid receiver, the reactor and a liquid separator. The reaction temperature was adjusted via a heat carrier-oil circuit. The pressure and hydrogen stream were controlled electronically. The 4,4′-HMDA-containing solution with methanol as solvent was added to the hydrogen stream using a pump and the mixture was delivered at the top of the reactor (trickle bed mode of operation). After the solution had trickled through the reactor, the product was removed from the separator at regular intervals. The concentration of the crude HMDA solution was 20 wt. % in all cases. The crude MDA contained 85 wt. % of 4,4′-HMDA. The crude HMDA contained a further 10 to 20 wt. % of oligomers. The reactor pressure was 80 bar in all cases, and the liquid load LHSV 0.5 h⁻¹. Ru-support catalyst from example 1 was used. The reaction temperature was 100° C.

The 4,4′-MDA conversion was 94%, the yield of 4,4′-HMDA 67%. The trans-trans-content was 14%.

### COMPARISON EXAMPLE 3

As in example 1, 4,4′-MDA was hydrogenated to 4,4′-HMDA at 100° C. using the Ru/Al₂O₃ supported catalyst not in accordance with the invention. The conversion of 4,4′-MDA was 84% and the yield of 4,4′-HMDA 59%. The trans-trans- content was 21%.

### EXAMPLE 4

3-hydroxypropanal was hydrogenated to 1,3-propanediol. Hydrogenation was carried out in the presence of the Ru/TiO₂ supported catalyst from example 2 according to the invention in the above-described hydrogenation apparatus. An aqueous solution containing 10 wt. % of 3-hydroxypropionic aldehyde and having a pH of 4.0 was used. The catalyst volume was 36 ml, the LHSV value 2 h⁻¹ and the reaction temperature 60° C. The selectivity of 1,3-propanediol was over 98%; n-propanol was a by-product. n-propanol formation was at 0.3 mol. %, based on 3-hydroxypropanal used (=HPA), after an operating period of 100 hours. The catalyst activity was 8.1 g of HPA per g of Ru per hour.

### Comparison Example 4

3-hydroxypropanal was hydrogenated as in example [sic], a Ru-rutile/anatase support catalyst from comparison example 2, not in accordance with the invention, being used. The hydrogenation apparatus, the catalyst volume and the hydrogenation conditions corresponded to the values in example 4 of the invention. The catalyst activity was 8.8 g of HPA per g of Ru per hour, but the formation of by-products—n-propanol—increased during the operating period and was 1.1 mol. %, based on 3-hydroxypropanal used, after an operating period of 100 hours.

Further variations and modifications of the foregoing will be apparent to those skilled in the art and are intended to be encompassed by the claims appended hereto. German priority application 100 54 347.2 is relied on and incorporated herein by reference.

We claim:

1. A process for the catalytic hydrogenation of an organic compound comprising subjecting said compound to hydrogen, in the presence of a supported catalyst with a coating containing ruthenium as a support-bound active metal and a total of 1.01 to 30 wt. % of support-bound active metals, wherein said supported catalyst has a support which is an oxide, carbide, nitride or siliceous support material having a BET (N₂) surface area smaller than 10 m²/g prior to loading with at least one active metal and the ruthenium content thereof makes up at least 50 wt. % of the active metals, with the proviso that diatomaceous earth with a BET (N₂) surface area greater than 2 m²/g is excluded.

2. The process according to claim 1 wherein the compound is a liable organic compound.

3. The process according to claim 1, wherein the ruthenium makes up at least 90 wt. % of the support-bound active metals.

4. The process according to claim 1, wherein the ruthenium makes up at least 99 wt. % of the support-bound active metals.

5. The process according to claim 1, wherein the supported catalyst contains one or more metals from the series of the 1st, 7th and 8th subsidiary group of the Periodic Table of Elements as active metals in addition to ruthenium.

6. The process according to claim 1, wherein the support material of the supported catalyst has a BET (N₂) surface area of 0.1 to smaller than 5 m²/g.

7. The process according to claim 6, wherein the support material of the supported catalyst has a BET (N₂) surface area in the range of 0.1 to 2 m²/g.

8. The process according to claim 1, the supported catalyst to be used contains 0.1 to 5 wt. %, of ruthenium.

9. The process according to claim 1, the supported catalyst to be used contains 0.5 to 3 wt. %, of ruthenium.

10. The process according to claim 1, wherein said compound is a compound selected from the group consisting of aromatic and heteroaromatic amines, nitrites and carbonyl compounds, phenols, aliphatic and cycloaliphatic carbonyl compounds, and the aliphatic and cycloaliphatic nitrites,

11. The process according to claim 1, wherein said compound is a hydroxy carbonyl compound.

12. The process according to claim 1, wherein said compound is a nitrile or carbonyl compound containing a further functional group in the α-, β- or γ-position.

13. The process according to claim 10, wherein a hydroxyaldehyde, as well as sugar is hydrogenated.

14. The process according to claim 10, wherein 3-hydroxypropionic aldehyde is hydrogenated.

15. The process according to claim 10, wherein 4,4′- or 4,2′-methylene dianiline or an isomer mixture thereof is hydrogenated as aromatic amine, 4,4′- or 4,2′-diaminodicyclohexylmethane or a reaction mixture containing this product being obtained.

16. A supported catalyst based on an oxide, carbide, siliceous or nitride support material and a coating containing at least ruthenium as active metal and a total of 0.01 to 30 wt. % of active metals, wherein the support material has a BET (N₂) surface area smaller than 10 m²/g prior to loading with at least one active metal and in where ruthenium makes up at least 50% of the active metals, with the proviso that diatomaceous earth with a BET (N₂)surface area greater than 2 m²/g is excluded.

17. The supported catalyst according to claim 16, wherein the uncoated support material has a BET (N₂) surface area of 0.1 to 5 m²/g and the active metal content of the supported catalyst is 0.1 to 5 wt. %, at least 90% being ruthenium.

18. The supported catalyst according to claim 16, wherein the BET (N₂) surface area is 0.1 to 2 m²/g.
